(19) United States
(12) Patent Application Publication
(10) Pub. No.: US 2020/0276107 A1
(43) Pub. Date: Sep. 3, 2020

(54) Treatment of herpes simplex symptoms on skin and mucous membrane of mammals

(71) Applicant: Jyri Nieminen, Escaldes-Engordany (AD)

(72) Inventors: Jyri Nieminen, Escaldes-Engordany (AD); Juha-Pekka Pöyry, Luxembourg (LU)

(21) Appl. No.: 16/494,789

(22) PCT Filed: Mar. 19, 2018
(86) PCT No.: PCT/FI2018/050202

§ 371 Date: Sep. 17, 2019

(30) Foreign Application Priority Data
Mar. 17, 2017 (FI) 20175246

(51) Int. Cl. A61K 9/00; A61K 9/06; A61P 31/22; A61K 33/38; A61K 31/785; A61K 47/18; A61K 47/22

(52) U.S. Cl. CPC A61K 9/0014; A61K 9/06; A61P 31/22; A61K 45/06; A61K 31/785; A61K 47/18; A61K 47/22; A61K 33/38

(57) ABSTRACT

Antiviral compositions, method of manufacturing such compositions and uses of the composition. The present composition is intended for use as an antiviral medicament for topical application on areas of the mammal skin or mucous membranes affected by a herpes virus, in particular herpes simplex virus. The composition comprises silver ions complexed with polyamine polymers, optionally in combination with other antiviral agent. By the present invention, herpes cold sores can be successfully treated or outbreaks thereof even prevented.

### TECHNICAL FIELD

The present invention relates to medical treatment of herpes symptoms, in particular herpes simplex symptoms, such as sores, on skin and mucous membrane of mammals by topical application of a medical composition. The invention also relates to antiviral compositions and to method of manufacturing such compositions.

### BACKGROUND

There are two types of herpes simplex virus. HSV-1 causes cold sores on the lips and on mucous membrane of the mouth. HSV-1 can also cause sores around the genitals. HSV-2 causes sores in the genital area. Globally approximately two-thirds of people under the age of 50 have herpes, according to WHO. The highest rates are found in Africa, South-East Asia and the Western Pacific, but nearly 60 percent of U.S. men and women between the ages of 14 and 49 are carrying the HSV-1 virus. About 16 to 25 percent of U.S. men and women between 14 and 49 are infected with HSV-2. Herpes is a lifelong infection and makes infected 2-3 times more likely to acquire HIV. The herpes infection is also often called fever blisters, cold sores, herpes catarrhalis, herpes facialis, herpes febrilis, herpes genitalis, herpes labialis, herpes mentalis, herpes preputialis, and herpes progenitalis.

According to WHO, herpes virus is a major global problem and current medicines are not working properly. During the last two decades, state of the art antiviral medication has been acyclovir, valacyclovir and similar, which affect slowly, and in topical treatments 5 to 10 days are needed for healing. Slow healing of outbreaks is a big problem due to pain, social shame, possibility to infect other people and possible secondary bacterial infections. Due to low bioavailability of common antiviral acyclovir, multiple and repetitive applications per day are needed, such as five pills per day for five days or applying of ointment 5-6 times a day. Acyclovir, valacyclovir and related antivirals are not efficient enough to provide a quick relief of the pain, burning, tingling and itching, and it takes several days to have an effect.

In addition to numerous acyclovir and valacyclovir related patents, various other treatments of herpes simplex have been proposed. U.S. Pat. No. 4,147,803 discloses the topical application of lauric diethanolamide to the area affected. Application of a mixture of boric acid, tannic acid, and salicylic acid is taught in U.S. Pat. No. 4,285,934. The use of lignosulfonate as a topical treating agent is disclosed in U.S. Pat. No. 4,185,097, and the application of kelp to the affected area is proposed in U.S. Pat. No. 4,117,120.

There is a significant, unmet need for new approaches for suppressing and treating herpes infections.

### SUMMARY OF THE INVENTION

It is an object of the present invention to provide a new composition for use in a method of treating herpes simplex in mammals.

It is another object of the invention to provide a novel method of treating herpes simplex virus infections in mammals.

The present invention is based on the concept of preparing a pharmaceutical topical composition for treating infections caused by herpes virus, comprising ionic silver present in the form of a polymeric complex.

The composition can be applied topically on the skin of a subject who suffers from an infection caused by a herpes virus, in particular a herpes simplex virus.

Thus, in a first aspect, there is provided a pharmaceutical topical composition for use as an antiviral medicament comprising a polyamine polymer complexing ionic silver.

In a second aspect, there is provided a pharmaceutical topical composition comprising a virucidal molecule like acyclovir or valacyclovir or similar and polyamine polymer complexing ionic silver. Different action mechanisms are having synergetic effects.

In another aspect, there is provided a method of treating a herpes virus infection in a subject. The method includes administering an effective amount of the pharmaceutical composition or formulation as disclosed herein.

More specifically, the compositions according to the present invention are characterized by what is stated in the characterizing part of claim 1.

The method according to the invention is characterized by what is stated in the characterizing part of claim 14.

Considerable advantages are obtained by the invention. Thus, the present invention helps to prevent herpes cold sores or even prevents outbreaks thereof. It shortens healing time of cold sores/outbreaks, provides a quick relief for the pain, burning and itching of cold sores/outbreaks and limits the risk of transmission of the infection to others or exposure to a secondary infection.

As will be discussed above, disappearance of symptoms including cold sores and lesions will be achieved within about 2 to 3 days, and relief of pain from the affected area, e.g. blisters, will be achieved within 10 to 15 minutes of the first application depending upon the size of the lesions or cold sores.

These and other objects and advantages of the invention will be discussed in connection with embodiments of the present invention, discussed in the following.

### EMBODIMENTS

In the present context, the abbreviation “HSV” is used for denoting herpes simplex and “HSV-1” for herpes simplex virus type I and “HSV-2” for herpes simplex virus type II. Further, “VZZ” stands for varicella zoster virus, “EBV” for epstein-barr virus and “CMV” and “hCMV” for cytomegalovirus and for human cytomegalovirus, respectively. “HPV” stands for human papilloma virus.

According to the present invention, symptoms caused by virus, such as HSV-1 and HSV-2 symptoms, for example cold sores, can be treated on skin and mucous membrane with silver polymer formulation of this innovation to solve the most of the drawbacks of today's state of the art medications.

Antimicrobial ionomer compositions comprising amine functional cationic polymer and ionic silver have been disclosed in WO 2013/026961 A1. Said publication is silent about the potential use of the compositions for antiviral therapy.

Surprisingly, silver polymer formulations as disclosed herein heal cold sores and outbreaks in 2 to 3 days and when tickling areas are treated before formation of cold sores, surprisingly the cold sores are not breaking out. Formulation gives fast and remarkable relief for itching and pain of cold sores. Silver polymer formulation is shown to be highly antiviral in in vitro tests, and it is killing effectively viral viruses in cold sores in vivo. This surprising effectivity remarkably limits the suffering of subjects infected with herpes and the possibility to infect others.

This application relates to the treatment of herpes infection on skin or mucous membrane, occurring in humans and animals or mammals, and provides effective topical formulations, dosage forms and dosing regimens improvably effective for arresting and treating such skin outbreaks, especially those caused by hCMV, HSV-1, HSV-2. Infections to be treated include genital herpes, herpes zoster, chicken pox and warts (HPV). The skin, eye, ear, nose, mouth and mucosal applications can be targeted with these formulations.

In a first aspect, there is provided a pharmaceutical topical composition comprising a polyamine polymer complexing ionic silver.

In a preferred embodiment of the invention, said polyamine polymer comprises a branched polyethyleneimine, a linear polyethyleneimine or a mixture of corresponding polyethyleneimines of different qualities with different properties with regard to e.g. molecular weights and primary:secondary:tertiary -amine ratios. Also copolymers of polyethyleneimines are useful.

The polyamine polymer preferably comprises or consists of a potentially branched poly(ethyleneimine) having a molecular weight (Mw) between 200 and 3,000,000, in particular about 750 to 2,000,000.

In a preferred embodiment of the invention, the silver ion source is silver halide, silver oxide, silver nitrate or metallic silver. In some embodiments, the silver ion source may be silver acetate, silver stearate, silver saccharinate, silver imidazole, silver citrate, silver methacrylate and silver sulfadiazine.

In a further preferred embodiment of the invention, the pharmaceutical topical composition further comprises a solvent, a solvent mixture, or a solvent matrix. The solvent is preferably a hydroxyl group containing solvent, such as water, ethanol, 1,3-butanediol, methyl, propyl or butyl alcohol or combinations thereof.

In a second aspect, there is provided a pharmaceutical topical composition comprising a virucidal molecule like acyclovir or valacyclovir or similar and polyamine polymer complexing ionic silver. Different action mechanisms are having synergetic effects.

In another aspect, there is provided a method of treating a herpes virus infection in a subject. The method includes administering an effective amount of the pharmaceutical composition or formulation as disclosed herein.

It has been found that herpes infections, in particular herpes simplex infections, can be effectively treated by topical application to the affected area of an effective amount of a composition comprising polyamine polymer complexing ionic silver. It has been found that application of 0.01 to 1 ml of polyamine polymer complex containing about 0.5 to 1.0 mg of ionic silver is an effective amount. The polyamine polymer silver complex is preferably diluted in a solvent or solvent mixture comprising hydroxy containing solvents like butyl glycol, ethanol etc. In addition to the active ingredients mentioned above the composition may contain a further antiviral substance, for example acyclovir, valacyclovir, penciclovir, famciclovir or similar first-line antiviral agent or combinations thereof.

The composition of this invention contains 10 to 100,000 ppm of ionic silver, and about 100 to 999,000 ppm of polyamine polymer, more preferably 100 to 10,000 ppm of ionic silver and 500 to 100,000 ppm of polyamine polymer. In a further embodiment the composition contains less than 7500 ppm of ionic silver.

The formulation may further contain a stabiliser component like saccharine or aminomethyl propanol.

The topical compositions of the invention can be for example in the form of cream, ointment, lotion, liquid, fluid, spray or foam, having a silver concentration of for example 5-1000 ppm. The pH of the formulation is preferably from 7.5 to 11.5.

The treatment of the herpes simplex infection in accordance with this invention comprises topical application of the composition disclosed herein to the affected area on the person suffering from the infection.

Generally, disappearance of symptoms including cold sores and lesions will be achieved within about 2 to 3 days. The relief of pain from the affected area will be achieved within 10 to 15 minutes after the first application depending upon the size of the lesions or cold sores when treatment is started.

In one embodiment, an effective amount of the composition 0.05 to 0.10 ml, containing about 0.50 to 1.00 mg of ionic silver per application, is applied to the affected area 1 to 4 times a day until healing is achieved.

Typically the area treated is about 0.01 to 200 cm², for example 0.05 to 100 cm², in particular about 0.1 to 50 cm², such as 0.15 to 10 cm² or 0.2 to 5 cm², or 0.25 to 2 cm² and the applied amount of ionic silver is about 0.01 to 10 mg/cm², for example about 0.1 to 5 mg/cm², typically per application.

The composition in accordance with the present invention is effective in the treatment of all areas on the mammal body affected by a herpes virus, for example herpes simplex virus, such as cold sores, lesions, warts and blisters. Mucous membranes in mouth and genital area are more sensitive compared skin and lips, and lower polyamine polymer silver ion complex concentrations can be effectively used to minimize irritation of mucous membrane.

The following non-limiting examples represent interesting embodiments.

### EXAMPLE 1

An antiviral composition was produced in a solvent matrix. 2.0 grams of a branched polyethyleneimine (Lupasol WF, BASF, MW 25000) was solubilized in 8 grams of 1,3-butanediol and cooled down. The solution was reacted together with 0.295 grams of silver chloride by mixing said suspension at room temperature until a clear solution was formed. The obtained ionomer composition had a dry content (mass of the solvent excluded) of 22.3% (w/w) and theoretical silver content of 9.7% (w/w) of dry mass.

### EXAMPLE 2

An antiviral ionomer composition was produced in a solvent matrix. Approximately 2.0 g of a branched polyethyleneimine (Lupasol G20 waterfree, BASF, molecular weight 1300) was mixed with 6 g of ethyl alcohol and cooled down. The solution was reacted with 0.724 g of silver saccharinate by mixing said suspension at room temperature until a clear solution was formed. The process was continued by adding of 2.49 ml of 1 M hydrochloric acid into said solution under continuous mixing. A clear solution of an optically clear ionomer composition was formed, having a dry content (w/w) of 25.0% and theoretical silver content of 9.5% (w/w) of dry mass.

### EXAMPLE 3

An antiviral ionomer composition was produced in a solvent matrix. 1.0 grams of a branched polyethyleneimine (Lupasol G20 waterfree, BASF, molecular weight 1300) was solubilized to 3 grams of ethyl alcohol and cooled down. The solution was reacted together with 0.156 grams of silver saccharinate by mixing said suspension at room temperature until a clear solution was formed. The process was continued by diluting the intermediate composition to a total volume of 50 ml with EtOH. Finally, the product was chloridized by adding 0.570 ml of 1 M HCl under mixing conditions.

### EXAMPLE 4

An antiviral ionomer composition was produced in a solvent matrix. 3.0 grams of a branched polyethyleneimine (Lupasol PS, BASF, molecular weight 750,000, concentration in water 33%) was mixed with 1 grams of ethyl alcohol and cooled down. The solution was reacted together with 0.156 grams of silver saccharinate by mixing said suspension at room temperature until a clear solution was formed. The intermediate composition was diluted to a total volume of 50 ml with EtOH. Finally, the composition was chloridized by adding 0.570 ml of 1 M HCl under mixing conditions.

### EXAMPLE 5

An antiviral ionomer composition was produced in a solvent matrix. 2.0 grams of a branched polyethyleneimine (Lupasol WF, BASF, MW 25,000) was solubilized in 8 grams of ethyl alcohol and cooled down. The solution was reacted together with 0.295 grams of silver chloride by mixing said suspension at room temperature until a clear solution was formed. The process was continued by adding 0.378 grams of saccharin and mixing until a clear solution was formed.

### EXAMPLE 6

An antiviral ionomer composition was produced in a solvent matrix. 2.0 grams of a branched polyethyleneimine (Lupasol P, BASF, MW 750,000, concentration in water 50%) was mixed with 5.460 grams of ethyl alcohol and cooled down. The solution was reacted together with 0.345 grams of silver chloride by mixing said suspension at room temperature until a clear solution was formed. The process was continued by adding 0.5 grams of saccharin. An ionomer composition with 14.1% w/w theoretical silver content (of dry weight) was produced.

### APPLICATION EXAMPLE 7

Antiviral performance of the composition produced as described in Example 1 was screened in vitro according to the European Standard EN 14476:2013 for a herpes simplex virus type 1 (HSV-1). Said composition was added to a suspension of herpes viruses in an interfering substance (clean conditions) in concentrations of 80% and 97%. The mixture was maintained at 20° C. for 60 seconds and 5 min. At the end of the exposure time, aliquots were taken, and the virucidal action was neutralised by transferring the aliquots into cold diluents. Serial dilutions were performed and assayed for residual infectivity. All viruses were found dead.

### APPLICATION EXAMPLE 8

Antiviral performance of the composition of the present invention was evaluated in vivo. The test group A consisted of four individuals suffering from herpes simplex labialis. All members of the group had a long history with herpes infection and all members had been testing all available herpes medications in the market. As infections were untreated, the individuals were normally healing in between 7 to 10 days. The time from initiation of the symptoms to when the patients experienced relief of pain from the affected area was between 5 to 7 days. As infections were treated with common topical acyclovir medication 5 to 6 times per day, healing resulted in between 5 to 7 days. The time from initiation of the symptoms to time when the patients experienced relief of pain from the affected area was 3 to 5 days. As infections were treated with common topical valacyclovir medication, recovery was similar compared acyclovir.

The herpes infections of test group were treated topically with a composition containing the polyamine polymer silver complex of the present invention with the following results. The composition was applied topically to the affected areas to cover the cold sores with a coating of the composition two times a day. The time from initiation of the cold sores and treatment with the composition to complete healing averaged between about 2 to 3 days. The time from the initiation of the treatment to when the patients experienced relief of pain from the affected area was about 10 to 15 minutes after the first application. As the composition of the present invention was applied topically to the affected areas as the first sensations of herpes outbreak appears, the cold sores didn't appear at all. The composition of present invention surprisingly prevents cold sores to develop when formulation is applied as the first sensations of outbreak appear.

### APPLICATION EXAMPLE 9

In another in vivo test, the test group B consisted of three individuals suffering from oral herpes simplex. As infections were untreated, the individuals resulted in complete healing normally in 8 to 11 days. The time from initiation of the symptoms to when the patients experienced relief of pain from the affected area was normally 6 to 7 days.

As the infections of test group B were treated topically with a composition containing the polyamine polymer silver complex of the present invention with the following results. The composition was applied topically to the cold sores to cover the affected areas with a layer of the composition two times a day. The time from initiation of the cold sores and treatment with the composition to healing was about two days. The time from the initiation of the treatment to when the patients experienced relief of pain from the affected area was about 10 to 15 minutes.

The composition of the invention is reducing the average time for healing about 70% compared to untreated herpes infections. More significantly, treatment with the composition of this invention results in relief of pain from the affected areas in about 500 to 1000 times faster than in untreated infections. More significantly, the composition of present invention surprisingly prevents cold sores or lesions to appear as formulation is applied at the first signs of tingling, burning or itching. More significantly, any member of test group A or B were not willing to stop using of the composition of the invention after the test period.

In vivo testing has shown that the compositions of the present invention are effective within the broad range of concentrations. The preferred range of concentration for silver is 100 ppm to 100000 ppm, more preferably 300 to 7500 ppm.

The compositions of the present invention can be used together with common antiviral molecules like acyclovir or valacyclovir or similar. Polyamine polymer silver ion complex is boosting performance of antiviral molecules remarkably. PEI is suitable carrier or delivery mechanism for acyclovir. PEI is distributing acyclovir molecules perfectly due to attraction forces between PEI molecule and capsid of herpes virus.

### INDUSTRIAL APPLICABILITY

The present compositions can be used for treatment of herpes simplex symptoms on skin and mucous membranes of mammals, such as humans, by topical application of a medical composition. The treatment can be combined with other forms of therapy and with the use of first-line and second line antiviral agents for treatment of, in particular HSV-1 and HSV-2 infections and with symptoms caused by such infections. Other virus infections which can be treated with the present invention include infections caused by CMV, hCMV, VZZ, EBV and HPV. As discussed above, the compositions can be formulated for application on skin, eye, ear, nose, mouth and mucous membranes.

### CITATION LIST

U.S. Pat. No. 4,147,803

U.S. Pat. No. 4,285,934

U.S. Pat. No. 4,185,097

U.S. Pat. No. 4,117,120

1. A composition for use as an antiviral medicament for topical application to areas on the mammal skin or mucous membrane affected with a herpes virus, said composition comprising silver ions complexed with a polyamine polymer.

2. The composition as claimed in claim 1, wherein the polyamine polymer comprises polyethyleneimine.

3. The composition according to claim 1, wherein a source of the silver ions is selected from the group of silver halides, silver oxides, silver nitrates, metallic silvers, and combinations thereof.

4. The composition according to claim 1, wherein the composition further comprises a solvent or a solvent mixture.

5. The composition according to claim 1, wherein the solvent comprises hydroxy groups.

6. The composition according to claim 1, wherein the silver concentration in the composition is 10-100,000 ppm.

7. The composition according to claim 1, wherein the composition further comprises a stabilizer component, and wherein the stabilizer component comprises saccharine, aminomethyl propanol, or a mixture thereof.

8. The composition according to claim 1, wherein a concentration of the polymer in the composition is between 1,000 and 100,000 ppm.

9. The composition according to claim 1, wherein the composition further comprises an additional antiviral component.

10. The composition according to claim 1, wherein a pH of the composition is 7.5 to 11.5.

11. The composition according to claim 1, wherein a pH of the composition is adjusted with hydrochloric acid.

12. The composition according to claim 1, wherein the composition comprises an ionomer composition of the silver ions and polyethyleneimine, the composition having a pH of 7.5 to 11.5 and containing 1000 to 100,000 ppm of silver.

13. A method of producing an antiviral pharmaceutical composition comprising the steps of mixing silver ions complexed with a polyamine polymer with an antiviral agent.

14. The method according to claim 13, wherein the antiviral agent comprises a member from the group consisting of acyclovir, valacyclovir, penciclovir, famciclovir, and combinations thereof.

15. A method of treating a herpes simplex virus infection in a mammal, comprising topically administering an effective amount of the composition according to claim 1 topically on areas of mammal skin or mucous membrane affected by a herpes simplex virus.

16. The method according to claim 15, wherein the effective amount of the composition is 0.05 to 0.10 ml, and comprises about 0.50 to 1.00 mg of ionic silver per application.

17. The method according to claim 14, wherein the herpes simplex virus is hCMV, HSV-1, or HSV-2.

18. The method according to claim 14, further comprising administering an additional antiviral component in conjunction with the component according to claim 1, wherein the additional antiviral component comprises a member selected from the group consisting of acyclovir valacyclovir, penciclovir, famciclovir, and combinations thereof,

19. The method according to claim 14, wherein the composition comprises an ionomer of silver chloride and branched polyethyleneimene.

20. The composition according to claim 1, wherein the additional antiviral component comprises a member selected from the group consisting of acyclovir, valacyclovir, penciclovir, famciclovir, and combinations thereof.
